(19) United States
(12) Patent Application Publication
(10) Pub. No.: US 2009/0306692 A1
(43) Pub. Date: Dec. 10, 2009

(54) ELECTROFORMED LIQUID JET SURGICAL INSTRUMENT

(75) Inventors: James E. Barrington, Lexington, MA (US); Kevin P. Staid, Lowell, MA (US)

Correspondence Address: MCCARTER & ENGLISH, LLP BOSTON, 265 Franklin Street, Boston, MA, 02110, US

(73) Assignee: HydroCision, Inc., N. Billerica, MA (US)

(21) Appl. No.: 12/298,638

(22) PCT Filed: Apr. 25, 2007
(86) PCT No.: PCT/US07/10040

§ 371 Date: Feb. 26, 2009

Related U.S. Application Data
(60) Provisional application No. 60/794,867, filed on Apr. 25, 2006.

(51) Int. Cl. A61B 17/3203; B29C 65/00; C25D 1/02

(52) U.S. Cl. 606/167; 156/150

(57) ABSTRACT

Certain embodiments of the invention provide a variety of methods of manufacturing a liquid jet-forming surgical instrument. According to these methods, a nozzle assembly of the instrument is electroformed on a mandrel. The nozzle assembly includes a nozzle providing a jet-opening, wherein the nozzle is shaped to form a liquid jet. In some embodiments, the mandrel includes a first mandrel portion and a second mandrel portion. Once the nozzle assembly is formed, the mandrel may be removed from the nozzle assembly. The nozzle assembly may in certain embodiments be coupled to an outlet of the pressure tube. In certain embodiments, an inlet of an evacuation tube is positioned such that a jet-receiving opening of the evacuation tube is positioned opposite the jet-opening of the nozzle.

### FIELD OF THE INVENTION

The invention relates generally to surgical instruments for creating a liquid jet and methods for making the instruments.

### BACKGROUND OF THE INVENTION

Liquid jet cutting instruments for industrial cutting operations are known, and have been adapted for smaller scale and more delicate procedures. In particular, certain such devices have been adapted for use in surgical procedures. Many changes are needed for successful adaptation of industrial cutting for surgical/medical use.

A variety of liquid jet instruments for surgery have been developed, including instruments described in commonly-owned U.S. Pat. No. 5,944,686, U.S. Pat. No. 6,375,635, U.S. Pat. No. 6,511,493, U.S. Pat. No. 6,451,017, U.S. Pat. No. 7,122,017, U.S. Pat. No. 6,960,182, U.S. Application Publication No. US2003-0125660, U.S. Application Publication No. US2002-0176788, U.S. Application Publication No. US2004-0228736, U.S. Application Publication No. US2004-0243157, U.S. Application Publication No. US2006-0264808, and U.S. Application Publication No. US2006-0229550, which are all incorporated by reference in their entireties.

While currently available surgical liquid jet instruments represent, in some instances, significant improvements over many prior art surgical instruments for performing open and minimally invasive surgical procedures, there remains a need in the art to provide improved methods of manufacturing liquid jet surgical instruments. The present invention provides, in many embodiments, improved methods of manufacturing various types of liquid jet forming surgical instruments. Certain embodiments are directed to methods of manufacturing a nozzle assembly for a liquid jet surgical instrument in a manner that may be easily reproducible.

### SUMMARY OF INVENTION

Several problems and manufacturing challenges have been determined and recognized within the context of the present invention. One of the major challenges in designing and manufacturing surgical instruments is that the instruments, or at least the parts of the system that contact the patient, are preferably disposable after the completion of the procedure. Because parts of the instrument may be disposable, it is advantageous for these components to be manufactured in a simple and repeatable way.

In addition, certain surgical instruments are configured to evacuate material away from the site of operation, by using suction, or, with certain liquid jet-forming surgical instruments, by using the stagnation pressure that can be generated by the passage of a high-velocity jet into a suitable evacuation tube without the need for additional suction (see, for example, commonly owned U.S. Pat. No. 6,375,635). Moreover, the site of operation can be in the interior of the body and may not be readily observable. Therefore, it may be important in a liquid jet-forming surgical instrument, for the jet emitting nozzle to be accurately aligned with the inlet opening of the evacuation tube.

A further challenge in manufacturing for medical use is that a 180-degree bend in the path of the high pressure fluid may be required for certain configurations, so that the liquid jet is directed back in the direction from which the liquid is supplied.

Another major challenge in making liquid jet instruments for medical and surgical use is the need for large scale manufacture of precisely and reproducibly dimensioned jet-forming nozzles or orifices, and the need to accurately and reproducibly align them within the instrument, while minimizing the number and complexity of manufacturing steps.

An improved method of manufacturing a liquid jet-forming surgical instrument by electroforming a nozzle assembly is provided. The nozzle assembly may be formed on a mandrel such that the outer surface of the mandrel forms the inner surface of the nozzle assembly. The mandrel may later be removed once the nozzle assembly is formed.

In one embodiment, a mandrel is inserted into an outlet of a pressure tube. At least a portion of the mandrel and the pressure tube may be coated with an electroconductive material, such as a metal, and then a nozzle assembly may be electroformed on the mandrel. After electroforming, portions of the nozzle assembly and/or the mandrel may be cut to create a jet-opening in the nozzle assembly. The mandrel may then be selectively removed.

In one embodiment, the nozzle assembly is integral with the pressure tube. An evacuation tube may be joined to the evacuation tube tube, either before or after the formation of the nozzle assembly, and the evacuation tube may be aligned so that the fluid jet emitted by jet-opening of the nozzle assembly will enter the lumen of the evacuation tube.

In another embodiment, a mandrel has a shape in a first region that will form a nozzle, after electroplating a layer on the mandrel and cutting it; and a shape in a second region that will form an opening that can be cut to expose the mandrel material so that, after cutting the layer and selectively removing the mandrel material, an opening will be formed which can fit onto or into a high-pressure tube.

In one aspect, the invention provides a method of manufacturing a liquid jet-forming surgical instrument comprising a pressure tube, an evacuation tube and a nozzle. A nozzle assembly of the surgical instrument is electroformed on a mandrel, where the nozzle assembly includes at least one nozzle providing a jet-opening, and the nozzle is shaped to form a liquid jet as a liquid at high pressure flows therethrough. The mandrel is removed from the nozzle assembly, and the outlet of the pressure tube of the surgical instrument is coupled to the nozzle assembly. An inlet of the evacuation tube of the surgical instrument is positioned such that a jet-receiving opening of the evacuation tube is located opposite the jet-opening of the nozzle to enable the evacuation opening to receive the liquid jet, when the instrument is in operation.

In another aspect, the invention provides a method of manufacturing a liquid jet-forming surgical instrument comprising a pressure tube, an evacuation tube and a nozzle. An outlet of the pressure tube of the surgical instrument is coupled to a mandrel, and a nozzle assembly of the surgical instrument is electroformed on the mandrel so that the nozzle assembly is integrally connected to the outlet of the pressure tube, where the nozzle assembly includes at least one nozzle providing a jet-opening. The nozzle is shaped to form a liquid jet as a liquid at high pressure flows therethrough. The mandrel is removed from the nozzle assembly and an inlet of the evacuation tube of the surgical instrument is positioned such that a jet-receiving opening of the evacuation tube is located opposite the jet-opening of the nozzle to enable the evacuation opening to receive the liquid jet, when the instrument is in operation.

In another aspect, the invention provides a method of manufacturing a liquid jet-forming surgical instrument comprising a pressure tube, an evacuation tube and a nozzle. A first mandrel portion is coupled to an outlet of the pressure tube of the surgical instrument, and a second mandrel portion is coupled to an inlet of the evacuation tube of the surgical instrument, where the second mandrel portion is constructed to be coupled to the first mandrel portion. A nozzle assembly of the surgical instrument is electroformed on the first and second mandrel portions. The nozzle assembly is cut to create at least one nozzle providing a jet-opening, wherein the nozzle is shaped to form a liquid jet as a liquid at high pressure flows therethrough. A jet-receiving opening of the inlet of the evacuation tube is located opposite the jet-opening of the nozzle to enable the evacuation opening to receive the liquid jet, when the instrument is in operation. The first and second mandrel portions are removed from the nozzle assembly.

In yet another aspect, the invention provides a method of manufacturing a liquid jet-forming surgical instrument comprising a pressure tube, an evacuation tube and a nozzle. A first end of a substantially U-shaped mandrel is coupled to an outlet of the pressure tube of the surgical instrument. At least a portion of the substantially U-shaped mandrel and at least a portion of the pressure tube are coated with an electroconductive material. A nozzle assembly of the surgical instrument is electroformed on the mandrel, and the nozzle assembly is cut to create at least one nozzle providing a jet-opening, where the nozzle is shaped to form a liquid jet as a liquid at high pressure flows therethrough. An inlet of the evacuation tube of the surgical instrument is positioned such that the longitudinal axis of the evacuation tube is substantially parallel to the longitudinal axis of the pressure tube, and such that a jet-receiving opening of the evacuation tube is located opposite the jet-opening of the nozzle to enable the evacuation opening to receive the liquid jet, when the instrument is in operation. The substantially U-shaped mandrel is then removed from the nozzle assembly.

In yet another aspect, the invention provides a method of manufacturing a liquid jet-forming surgical instrument comprising a pressure tube, an evacuation tube and a nozzle. A nozzle assembly of the surgical instrument is electroformed on a mandrel, where the nozzle assembly includes at least one nozzle providing a jet-opening, and the nozzle is shaped to form a liquid jet as a liquid at high pressure flows therethrough. The mandrel is removed from the nozzle assembly and an outlet of the pressure tube of the surgical instrument is coupled to the nozzle assembly.

In yet another aspect, the invention provides a method of manufacturing a liquid jet-forming surgical instrument comprising a pressure tube and a nozzle. An outlet of the pressure tube of the surgical instrument is coupled to a mandrel, and a nozzle assembly of the surgical instrument is electroformed on the mandrel so that the nozzle assembly is integrally connected to the outlet of the pressure tube, where the nozzle assembly includes at least one nozzle providing a jet-opening. The nozzle is shaped to form a liquid jet as a liquid at high pressure flows therethrough. The mandrel is removed from the nozzle assembly.

### BRIEF DESCRIPTION OF DRAWINGS

The accompanying drawings are schematic and are not intended to be drawn to scale. In the figures, each identical, or substantially similar component that is illustrated in various figures is typically represented by a single numeral or notation. For purposes of clarity, not every component is labeled in every figure, nor is every component of each embodiment of the invention shown where illustration is not necessary to allow those of ordinary skill in the art to understand the invention. In the drawings:

FIG. 1A is a schematic cross-sectional illustration of a liquid jet-forming surgical instrument with an electroformed nozzle assembly;

FIG. 1B is a schematic side view illustration of the surgical instrument shown in FIG. 1A;

FIG. 1C is a detailed schematic cross-sectional illustration of the surgical instrument shown in FIG. 1A;

FIG. 2 is a schematic illustration of a method of manufacturing a liquid jet-forming surgical instrument according to one embodiment, in which an evacuation tube is coupled to a pressure tube, a mandrel is inserted into the pressure tube, and the mandrel and pressure tube are coated with an electroconductive material;

FIG. 3A is a schematic illustration of a mandrel according to one embodiment;

FIG. 3B is a schematic cross-sectional illustration of the mandrel shown in FIG. 3A;

FIGS. 3C and 3D are schematic end view illustrations of the mandrel shown in FIG. 3A;

FIG. 4A is a schematic end view illustration of a nozzle assembly electroformed on a mandrel;

FIG. 4B is a schematic cross-sectional illustration of the nozzle assembly electroformed on a mandrel coupled to a pressure tube;

FIG. 4C is a schematic illustration of an electroformed nozzle assembly according to one embodiment;

FIGS. 4D and 4E are schematic illustrations of an electroformed nozzle assembly according to another embodiment;

FIG. 5A is a schematic illustration of a second mandrel portion according to one embodiment;

FIG. 5B is a schematic end view illustration of the second mandrel portion shown in FIG. 5A;

FIG. 5C is a schematic side view illustration of the second mandrel portion shown in FIG. 5A;

FIG. 6A is a schematic illustration of a first and a second mandrel portion used to electroform a nozzle assembly according to one embodiment;

FIG. 6B is a schematic side view illustration of the first and second mandrel portions shown in FIG. 6A;

FIG. 6C is a schematic cross-sectional illustration of the first and second mandrel portions shown in FIG. 6A;

FIG. 6D is a detailed schematic cross-sectional illustration of the surgical instrument shown in FIG. 6C;

FIG. 7A is a schematic illustration of a nozzle assembly electroformed onto the first and second mandrel portions according to one embodiment;

FIG. 7B is schematic end view illustration of the nozzle assembly shown in FIG. 7A;

FIG. 7C is a schematic cross-sectional illustration of the nozzle assembly shown in FIG. 7A;

FIG. 8A is a schematic illustration of a tissue cutting surface according to one embodiment;

FIGS. 8B and 8C are schematic illustrations of the tissue cutting surface shown in FIG. 8A on a nozzle assembly;

FIG. 9 is a schematic illustration of an electroformed nozzle assembly according to another embodiment;

FIGS. 10A and 10B are schematic illustrations of a first and a second mandrel portion used to electroform a nozzle assembly according to another embodiment;

FIG. 11 is a schematic illustration of a first and a second mandrel portion used to electroform a nozzle assembly according to yet another embodiment;

FIG. 12A is a schematic cross-sectional illustration of a pressure tube and evacuation tube; and

FIG. 12B is a schematic cross-sectional illustration of a pressure tube and evacuation tube with auxiliary tubes according to another embodiment.

### DETAILED DESCRIPTION

Disclosed here are inventive methods for manufacturing a variety of liquid jet instruments useful in a variety of applications and a variety of inventive liquid jet instruments formed by the instruments. Certain embodiments of the inventive instruments are especially well suited for a variety of surgical procedures. Certain embodiments of the liquid jet instruments provided by the invention can be configured in a variety of different ways for use in various surgical operating fields. Certain surgical instruments, according to the invention, are configured as surgical handpieces having a proximal end with a grasping region, or handle, shaped and configured to be comfortably held by the hand of an operator. The instruments may also have a distal end that includes at least one nozzle for forming a liquid jet. The distal end of certain embodiments of the inventive surgical instruments can be used to perform a surgical procedure on a patient. The invention may also be practiced utilizing liquid jet instruments having a variety of configurations and purposes. Certain embodiments of the liquid jet instruments provided by the invention can be used in a wide variety of surgical applications to utilize a high pressure liquid stream to cut, drill, bore, perforate, strip, delaminate, liquefy, ablate, shape, or form various tissues, organs, etc. of the body of a patient.

It should be noted that a detailed treatment and discussion of a wide variety of design parameters, configurations, materials of construction, and other aspects of the design, fabrication, and construction of liquid jet surgical instruments useful for practicing various embodiments of the present invention are provided in commonly owned U.S. Pat. Nos. 5,944,686; 6,375,635; 6,511,493; 6,451,017; 7,122,017; and 6,960,182; in U.S. Patent Application Publication Nos. 2003/0125660 A1, US2002-0176788 A1, US2004-0228736 A1, 2004/0243157 A1, US2006-0264808 A1, and US2006-0229550, each of which is incorporated herein by reference. The reader is referred to these issued patents and patent publications for detailed description of and guidance as to the construction and design of certain embodiments of the liquid jet components of the instruments described herein.

Various embodiments of the invention are directed to liquid jet surgical instruments in which a nozzle assembly is electroformed on a mandrel. Electroforming is a process for fabricating a metal part by electrodeposition in a plating bath over a substrate or mandrel which is subsequently removed. A brief discussion of electroforming is provided below. However, it should be appreciated that methods and apparatus used to generally electroform a metal part, which are well known to those skilled in the art, are not described in detail herein, because the methods used for these processes are not materially different from the art.

The nozzle assembly may be located at or near the distal end of the surgical instrument, however, the invention is not limited in this respect. Furthermore, many of the below-described embodiments illustrate surgical instruments having a nozzle assembly which emits a jet in a proximal direction into an evacuation tube. However, the surgical instrument of the present invention may be configured differently, as the invention is not so limited. For example, the nozzle assembly may be configured to emit a jet in a distal direction or in a lateral direction. A variety of different designs with differing detailed specifications are contemplated, for a variety of uses. For example, the invention may be practiced in the manufacture of many of the wide variety of surgical liquid jet instrument configurations disclosed in commonly owned U.S. Pat. Nos. 5,944,686; 6,375,635; 6,511,493; 6,451,017; 7,122,017; and 6,960,182; in U.S. Patent Application Publication Nos. 2003/0125660 A1, US2002-0176788 A1, US2004-0228736 A1, 2004/0243157 A1, US2006-0264808 A1, and US2006-0229550.

Certain inventive surgical liquid jet instruments will now be described in more complete detail in the context of several specific embodiments illustrated in the appended figures. It is to be understood that the embodiments described are for illustrative purposes only and that the novel features of the invention, as described in the appended claims, can be practiced in other ways or utilized for instruments having other configurations, as apparent to those of ordinary skill in the art.

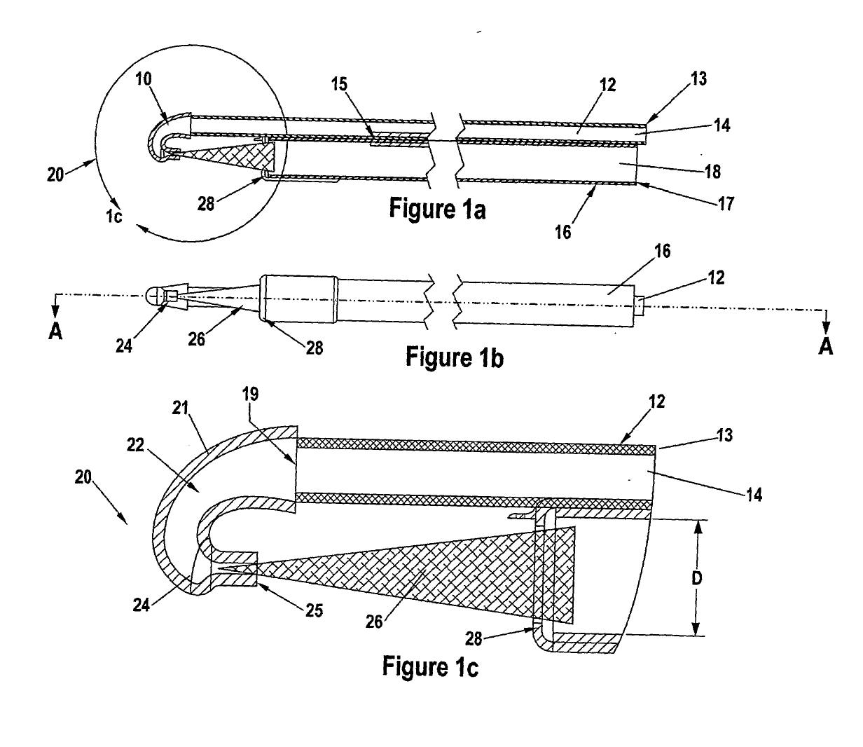

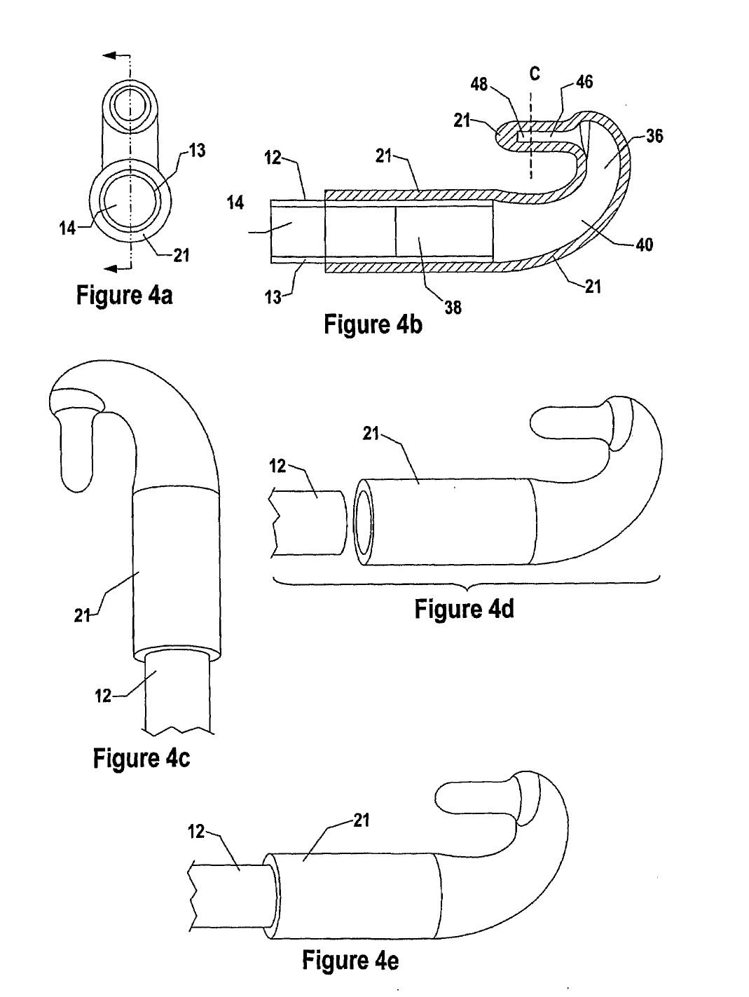

FIGS. 1A-1C illustrate one configuration of a liquid jet-forming surgical instrument according to an embodiment of the invention. More particularly, FIG. 1A illustrates a cross-sectional view of the instrument 10, FIG. 1B illustrates a side view of the instrument 10, and FIG. 1C illustrates a detailed view of tip region shown in FIG. 1A. As shown, in this embodiment, a fluid jet 26 is directed in a proximal direction. In these figures, the surgical instrument is shown generally as 10, and the tip region is shown generally as 20. Two tubes are shown, a pressure tube 12 having a wall 13 and a lumen 14, and an evacuation tube 16, having a wall 17 and a lumen 18. The electroformed nozzle assembly 21, having an interior volume 22 in fluid connection with the pressure tube lumen 14, is coupled to an outlet of the pressure tube 12. In one embodiment, the nozzle assembly 21 is electroformed on a portion of the pressure tube 12, e.g., an outlet portion. In another embodiment, the nozzle assembly 21 may be coupled to the pressure tube 12 by attachment after fabrication of the nozzle assembly 21 for example by soldering, welding, crimping, gluing, or other known connecting technique (e.g., see FIGS. 4D and 4E). As shown, in one embodiment, the nozzle assembly 21 is coupled to the distal end of the pressure tube 12, which, in the illustrated embodiment, comprises the outlet. In this illustrative embodiment, the pressure tube 12 is coupled to the evacuation tube 16 with pre-formed aligning connectors 15, which are rigidly attached to both the pressure tube and the evacuation tube. In another embodiment, the tubes 12, 16 may be welded together or otherwise permanently joined. It should be appreciated that in embodiments where the pressure tube 12 is coupled to the evacuation tube 16, the tubes may be coupled either before or after the electroformed nozzle assembly 21 is created. In yet other alternative embodiments the pressure tube and the evacuation tube are rigidly coupled and, in certain such embodiments, at least one of the tubes is free to move, longitudinally, laterally, and/or rotationally with respect to the other of the tubes. In certain such embodiments, such motion may be controllable by an operator of the instrument to facilitate insertion and deployment of the instrument, changes in cutting length, etc. (see e.g., U.S. Pat. No. 6,375,635).

As shown, the nozzle assembly 21 may include a collimated nozzle region 24 adjacent a jet-opening 25 (FIG. 1C). The nozzle assembly 21 may have an appropriate size and shape, as discussed further below, to form a fluid jet 26 through the jet-opening 25. Furthermore, the narrow, optionally converging collimated nozzle region 24 of the nozzle assembly can assist to collimate the fluid jet. The fluid jet 26 may diverge to some degree, depending on the geometry of the nozzle, length and shape of the collimating region, etc. (see e.g., U.S. Pat. No. 6,375,635), upon leaving the jet-opening 25, and may expand to a diameter D at the receiving opening 28 of the evacuation tube 16. The diameter D of the jet 26 may be the same as, or preferably somewhat less than, the diameter of the evacuation tube distal opening 28 at that point. In some embodiments, the jet receiving opening 28 of the evacuation tube 16 is smaller in diameter than the diameter of the lumen 18 in the rest of the evacuation tube 16. The diameter reduction at the opening 28 may act to create a venturi effect to entrain debris, as described in U.S. Pat. No. 6,375,635. The diameter reduction may also make the inlet of the evacuation tube less prone to cause trauma to tissues it contacts, as described in US 2006-0229550. Additionally, the reduced diameter of the evacuation tube 16 at its inlet 28 may help to prevent tissue from clogging the lumen 18, because tissue entering through limiting orifice 28 is small enough to pass through the rest of the evacuation system.

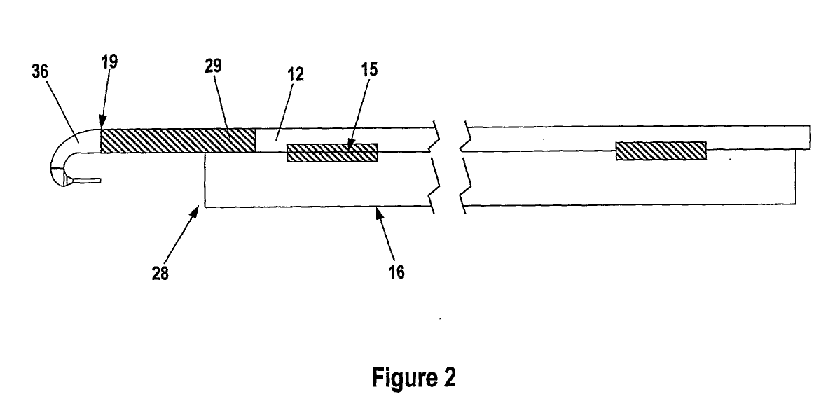

Turning now to FIG. 2, one inventive method of manufacturing a liquid jet-forming surgical instrument, such as that illustrated in FIGS. 1A-1C, will now be discussed in greater detail. As illustrated, according to one embodiment, a mandrel 36 (also referred to as a first mandrel portion 36) is coupled to the pressure tube 12. As shown, the mandrel 36 is inserted into the distal end 19 of pressure tube 12. In this embodiment, the evacuation tube 16 is also coupled to a pressure tube 12. In particular, the pressure tube 12 with distal tip 19 defining an outlet, and an evacuation tube 16 with an inlet 28, may be coupled together with the assistance of alignment connectors 15. Tubes 12, 16 may also be coupled together by welding, brazing, or similar methods, or may be held in alignment and proximity without rigid interconnection.

In one embodiment, the mandrel 36 is made of a thermoplastic material, such as polystyrene. In other embodiments, the mandrel 36 may be made of other materials, and any material which can be reliably removed in production, e.g., via heating/melting, dissolution, degradation, etc. is potentially suitable for use in the mandrel or mandrels of the invention. For example, the mandrel could be aluminum, and the removal procedure could be removal of the aluminum by etching with alkaline solutions. In another embodiment, a material used to form a mandrel 36 may be dissolved, for example without heating. In one embodiment, a wax can be a suitable mandrel material. As noted above, any mandrel removal method may be used in the invention that does not deleteriously alter the properties of the electroformed tip of the instrument. In certain embodiments, the mandrel 36 is solid, whereas in other embodiments, portions of the mandrel may be hollow, as the invention is not limited in this respect.

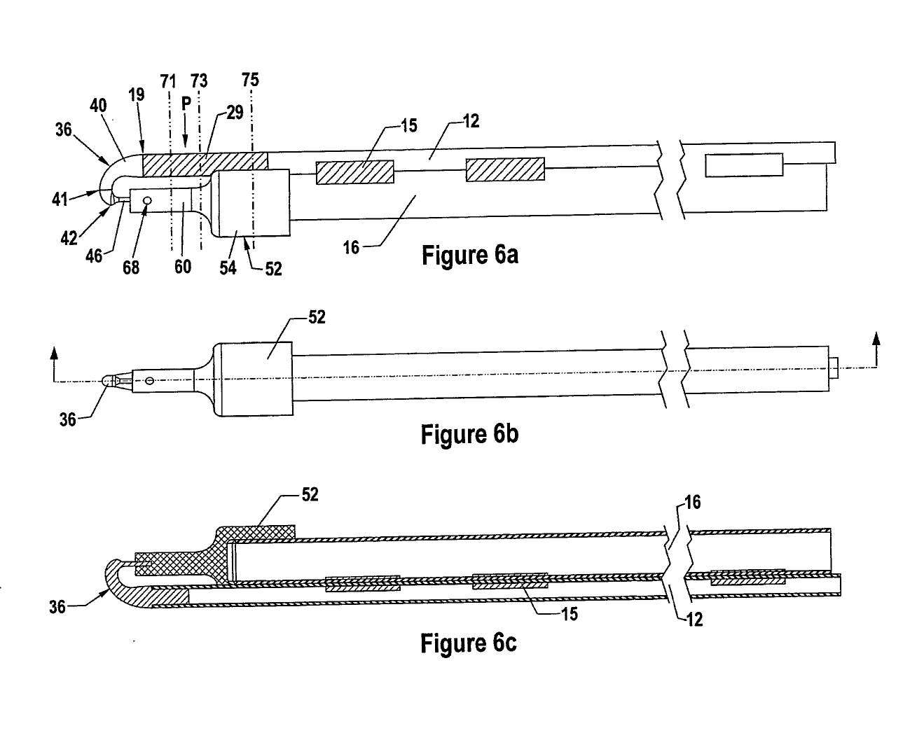

At least a portion of the mandrel 36 and at least a portion of the pressure tube 12, such as the terminal region 29 of tube 12 may be coated with an electroconductive material, such as gold, or the mandrel and the pressure tube otherwise may be made to be uniformly electroconductive before the electroforming occurs, when the nozzle assembly 21 (not shown in this figure because not yet formed) is created in situ on the tube 12. It should be recognized that an electroconductive material on tube 12 is not generally needed if the nozzle assembly 21 is electroformed separately and thereafter coupled to the pressure tube 12 after the fabrication of the nozzle assembly 21. The electroconductive coated region 29 may be long enough to overlap the inlet opening 28 of the evacuation tube 16, as shown. In other embodiments, the coated region 29 may be shorter, extending from the distal end 19 of the pressure tube 12 to a point “P” on the pressure tube 12 that is distal of the tip 28 of the evacuation tube 16 (see FIG. 6A).

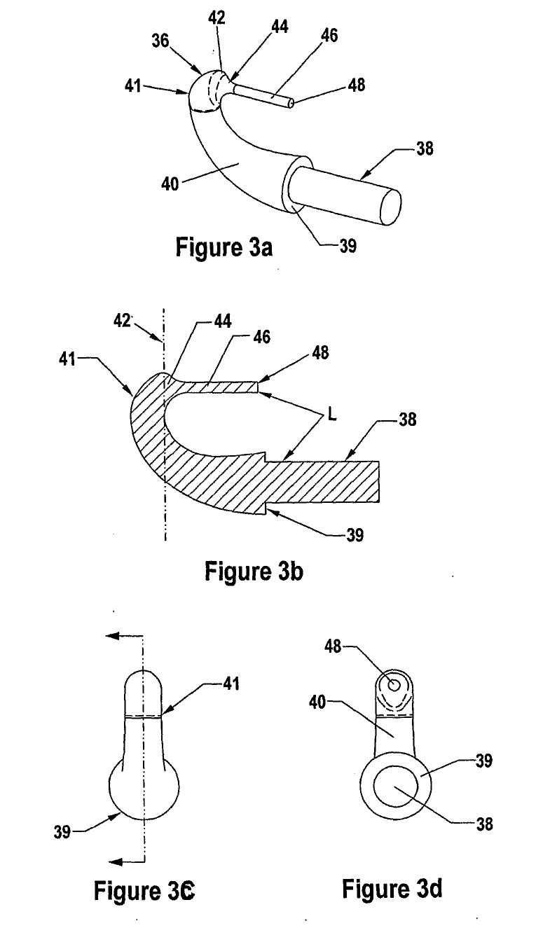

FIGS. 3A-3D illustrate the first mandrel portion 36 according to one embodiment. The first mandrel portion 36 is used for forming the electroformed nozzle assembly 21. In some embodiments, the first mandrel portion 36 is the only mandrel used to electroform a nozzle assembly 21. As discussed in greater detail below, in other embodiments, a plurality of mandrel portions may be used to electroform a nozzle assembly 21, as the invention is not limited in this respect. As seen in the perspective view (FIG. 3A) and cross section view (FIG. 3B), the mandrel 36 may include a post 38 constructed to fit closely in the outlet end 19 of the pressure tube 12. The post 38 may end in a shoulder 39, where the mandrel broadens out to a larger diameter. The larger mandrel diameter may be substantially equal to the outer diameter of the pressure tube 12. In one embodiment, the larger mandrel diameter is less than the outer diameter of the pressure tube 12 to minimize disturbance of flow through the finished nozzle assembly 21. In another embodiment, the mandrel 36 does not have a shoulder 39, and the post 38 may be glued, or otherwise reversibly adhered or reversibly mechanically fixed in place, inside of the pressure tube 12 so as to maintain an appropriate depth in the tube 12 during processing. In yet another embodiment, the shoulder 39 is a small bump, or is a small expansion of the diameter of central portion 40 (see below) compared to post region 38. In one embodiment, the post 38 is partially or substantially hollow, to make it easier to remove after the nozzle assembly 21 is electroformed.

In the embodiment illustrated in FIGS. 3A-3D, the mandrel 36 has a central portion 40 lying between the shoulder 39 and the tip end 48. As shown, the central portion 40 of the mandrel 36 may have curvature, and the diameter of the mandrel portion 40 may gradually decrease as the mandrel curves.

In one embodiment, as shown, the amount of curvature in the mandrel is approximately 180 degrees. In this orientation, the mandrel 36 may be configured to create a nozzle assembly 21 having a jet-opening such that liquid jet is directed through the jet-opening and in a proximal direction along the axis of the instrument 10. In other embodiments, the mandrel 36 may be configured differently, as the invention is not limited in this respect. For example, in one embodiment, the amount of curvature in the mandrel is at least approximately 145 degrees. In another embodiment, the amount of curvature in the mandrel is at least approximately 120 degrees, and in another embodiment, the amount of curvature in the mandrel is at least approximately 90 degrees, or 60 degrees.

In the embodiment illustrated in FIGS. 3A-3D, the distal tip of the mandrel 36 is located at 41. In a plane 42 perpendicular to the instrument axis and approximately tangent to the inside of the curved portion directly proximal of the distal extremity 41, the shape of the mandrel may change to a tapering shape, in the direction parallel to the direction of the device axis A--A of FIG. 1, over a tapering zone 44. This may be most easily seen in FIG. 3A. Beyond zone 44, in the embodiment illustrated, the mandrel nozzle region closely approximates a right circular cylinder and extends a selected distance proximally of the tapering zone 44 to a tip 48. The nozzle region 46 may be asymmetrical in relation to tapering zone 44, and the tapering zone 44 need not be rotationally symmetrical, although it is so shown in FIG. 3A. The length of the nozzle region 46 may vary, as the invention is not limited in this respect. Longer nozzle regions 46 may assist in collimating the jet beam 26 (see e.g., U.S. Pat. No. 6,375,635), but longer nozzle regions may also displace the active cutting zone so that it is more proximal of the distal extremity 41.

The mandrel 36 is designed to create an interior volume 22 of the nozzle assembly (FIGS. 1A-1C), after the nozzle assembly 21 is electroformed and the mandrel is removed. This volume 22 is generally smooth and may be gradually tapering. In some embodiments, the mandrel's 36 design is a precise matching of the pressure tube wall thickness 13 at the shoulder 39. In other embodiments, the sharp shoulder 39 may be replaced by a gradual widening. In yet other embodiments, the shoulder 39 is minimized to make the transition in the wall profile between the pressure tube 12 and the wall 21 of the nozzle assembly 21 as smooth as feasible. In yet other embodiments, the post 38 may be continuous with region 40, and may be approximately the same diameter as the outer diameter of pressure tube 12, or slightly larger, to allow the mandrel to be slid onto tube 12 and fastened in place.

In some embodiments, there is a close parallelism between the axis of the nozzle region 46 of the mandrel, and the axis of the post 38. In certain embodiments, there is less than a few degrees of deviation between these two axes. The substantially parallel sections are marked “L” in the embodiment shown in FIG. 3B. Because the nozzle region 46 can have a diameter below approximately 0.005 inch (0.125 mm), it may be demanding to maintain this degree of parallelism during production and mounting of the mandrel. The close parallelism may be important in this embodiment for placing the liquid jet 26 in reproducible and accurate alignment with the evacuation tube 16, as described further below.

While the mandrel illustrated in FIGS. 3A-3D is one illustrative embodiment, it should be appreciated that in other embodiments, the mandrel does not have to be smoothly tapered, or rotationally symmetric. One reason to have a smooth design is to prevent a large pressure drop in the nozzle assembly 21. In certain embodiments, the pressure drop may be less critical and thus a mandrel having a less smooth design is also contemplated. Furthermore, it is also contemplated that for certain applications, other, simpler, shapes may also be used which may be easier to prototype and manufacture:

To form the surgical instrument 10 shown in FIGS. 1A-1C, at least a portion of the mandrel 36 is subject to electroforming thereon to form the nozzle assembly 21, or a least a portion thereof. As mentioned above, electroforming is a process for fabricating a metal part by electrodeposition in a plating bath over a mandrel which is subsequently removed. A metal part is formed over the mandrel by controlling the electrodeposition of metal passing through an electrolytic solution onto a metal or metallized mandrel. A metal layer or skin is built up on the mandrel or any surface that has been rendered electroconductive through the application of a paint or coating that contains metal particles. Methods for electroforming generally, are well-developed and numerous commercially available service providers exist that will electroform parts to provided specifications. In certain embodiment of the present invention, the nozzle assembly 21 of the present invention was electroformed by the A. J. Tuck Company, located in Brookfield, Conn. Additional information regarding their electroforming process may be obtained from their website, www.ajtuckco.com.

In the electroforming process, an electrolytic bath is used to deposit nickel or other electroplatable metal onto a conductive mandrel surface. Once the plated material has been built up to the desired thickness, the electroformed part is taken off the mandrel or the mandrel is removed from the electroformed part. In one particular embodiment, the electroforming process continues until the thickness of the wall of the nozzle assembly is at least approximately 0.125 millimeters.

FIG. 4C illustrates the external appearance of the electroformed nozzle assembly, whereas FIGS. 4A and 4B illustrate the nozzle assembly 21 electroformed on a mandrel 36 coupled to the pressure tube 12. The nozzle assembly 21 covers the outlet region of pressure tube 12 and its tip 19, and the mandrel's distal regions 40, 44 and 46.

In one embodiment, before attempting to remove the mandrel 36, a cut is made at a selected plane C, here shown as perpendicular to the device axis, through both the electroformed nozzle assembly 21 and the mandrel's tip region 46 to expose a nozzle jet-opening 48. The nozzle assembly 21 begins to look more similar to the nozzle assembly 21 shown in FIGS. 1 and 2, after removal of this tip, and removal of the mandrel material. In this embodiment, the nozzle jet-opening has been accurately formed, and accurately aligned with the axis of the instrument, during the manufacturing process. It is possible to avoid expensive post-manufacturing alignment of the nozzle with the system of manufacture of the invention.

The removal of a mandrel, or the removal of a portion of a mandrel, after the formation of an electroformed nozzle assembly on the mandrel, may be made by any convenient process. As mentioned above, in one embodiment, the mandrel 36 is made of a thermoplastic material, such as polystyrene. In this embodiment, the tip area may be heated to about 430-475° F. (ca. 222-250° C.). This is well above the melting point of polystyrene, to reduce the viscosity of the thermoplastic mandrel. Either after warming or during it, one or several atmospheres of air pressure may be applied to force the melted plastic mandrel out of the tip. The device may be further cleaned with a suitable solvent, for example acetone, THF, methylene chloride or the like, which may occur at an elevated temperature, for example at 50-100° C.

An alternative method of fabrication is illustrated in FIGS. 4D and 4E. In this alternative method, instead of the mandrel being coupled to the pressure tube prior to electroforming the nozzle assembly integral with the outlet portion of the pressure tube, the nozzle assembly 21 is separately formed without the mandrel being coupled to the pressure tube 12. After removal of the mandrel, the stand-alone nozzle assembly is then coupled to the outlet of the pressure tube 12 (FIG. 4E) by any suitable method of attachment capable of withstanding the contemplated operating pressures (e.g., 1000 psig or greater), such as welding, brazing, gluing, crimping, press-fitting, solder fitting, electroforming, etc.

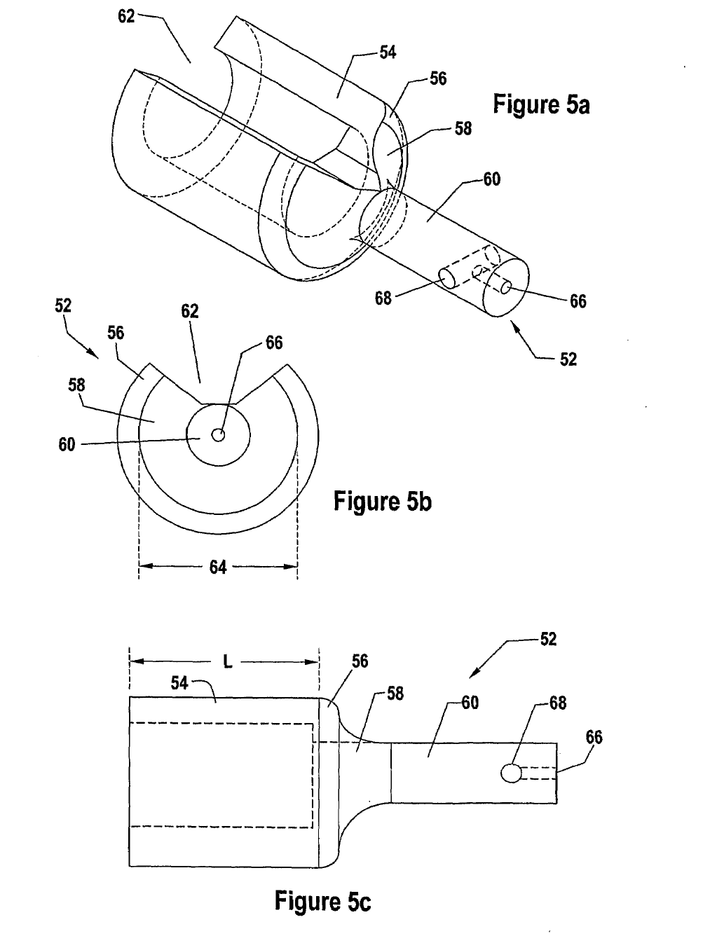

FIGS. 5A-5C illustrate a second mandrel portion 52 which, according to some embodiments, may be used in combination with the first mandrel portion 36. In some embodiments, such as that illustrated in FIGS. 5A-5C the evacuation and pressure tubes are coupled together before the nozzle assembly 21 is electroformed. In this respect, the two tubes 12, 16 may be aligned such that the placement of the jet-opening 25 (shown in FIG. 1C) with respect to the evacuation tube 16 may be achieved using a second mandrel portion 52. As shown in the projection view FIG. 5A and the end view of FIG. 5B, the second mandrel portion 52 has a hollow first section 54, a second, convex, transition section 56, a third, concave transition section 58, and a fourth straight tip section 60. The concave and convex regions may be annularly concave and convex, i.e., extending around the circumference of the second mandrel 52. The first section 54 is cut away on one side, leaving a cutaway 62 (most easily seen in FIG. 5A and FIG. 5C.) The design illustrated with the two segments 56 and 58 prevents the formation of a sharp corner in this region, which may be desirable, but in other embodiments, the profile need not be distinctly segmented, and may have a corner or sharp edge without compromising functionality.

The full inner diameter 64 of the hollow segment 54, shown in FIG. 5B, may be large enough to accommodate the evacuation tube 16. The cutaway 62 may be sized to accommodate the adjoining pressure tube 12, and the cutaway may also prevent rotation of the second mandrel portion 52 with respect to the tubes 12, 16. The clearance between the evacuation tube 16 and the inside diameter 64 of the second mandrel portion 52 may be as small as practicable, and the combination of the clearance and the length L of the hollow portion is sufficiently constraining to retain the parallelism between a longitudinal axis (not labeled) of the second mandrel portion 52, and the longitudinal axis of the evacuation tube 16 (as illustrated in FIG. 6). The second mandrel portion 52 may be configured to retain the evacuation tube 16, such that the axis of the evacuation tube 16 and the axis of the second mandrel portion 52 are parallel to within less than a few degrees, for example less than approximately 10 degrees in one embodiment, and less than approximately 5 degrees in another embodiment.

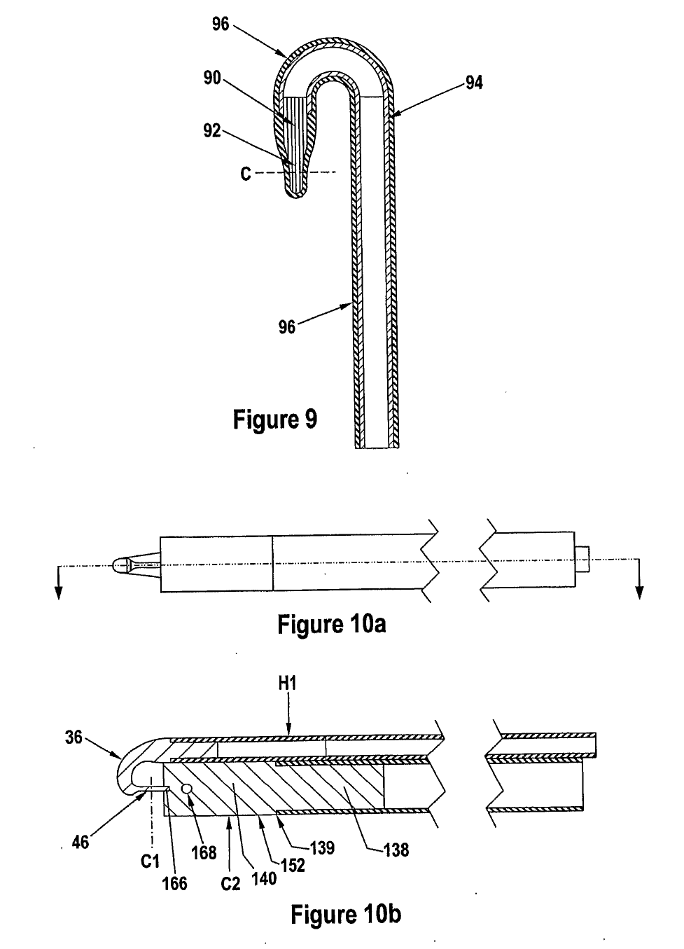

While in these examples the axis of the jet beam emitted by the jet-opening in the nozzle assembly, and the axis of the evacuation tube, are substantially parallel and essentially concentric, these features are not required for the practice of the invention. The jet beam need not be parallel with the evacuation tube, and the beam need not enter the evacuation tube concentrically. As discussed in greater detail below, in some embodiments, there may not be an evacuation tube (see FIG. 9 below, for example). It may be important simply to emit the jet beam in a controlled direction with respect to the instrument axis without needing to make post-fabrication adjustments. Examples of effective water jet surgical instruments with non-concentric and non-aligned axes are known, and are described for example in our copending application US2003-0125660A1. The predictability, the reproducibility, and the lack of need for post-fabrication alignment, help make the electroformed nozzle assembly of certain embodiments of the invention advantageous.

In FIGS. 5A-5C, the distal tip section 60 of second mandrel portion 52 has a first circular hole 66 concentric to the axis. The hole 66 may have a diameter sufficiently large to admit the tip 48 of the nozzle region 46 of the first mandrel 36, as shown in FIG. 3 or FIG. 4. In one embodiment, the fit should allow mating of the parts without force or distortion, but be a close enough fit to maintain the common alignment of the first and second mandrel portions 36 and 52 that is derived from the common orientation of the outer surfaces of tubes 12 and 16. The tip section 60 may also have a second hole 68 which may be perpendicular to the first hole 66 and intersecting with it, for removal of chips during machining of hole 66.

In one embodiment, the first and second mandrel portions 36, 52 are made by injection molding. Any material suitable for injection molding can be used, such as high impact polystyrene, provided that the material can be dissolved and/or melted and/or removed by etching or other conventional process, after electroforming the final shape onto the mandrel, in order to open up the inner volume 22 of the nozzle assembly 21. Suitable materials include, without limitation, plastic material that can be removed from the interior of the assembly by melting (and typically pressure ejecting the melt), and/or by solvent extraction. In many embodiments, the mandrel materials are also insoluble and non-swelling in electroplating solutions, and in certain embodiments, the mandrel materials do not melt below about 130° F. (about 55° C.). In certain embodiments, the material is also able to accept a conductive coating. In one embodiment, a selected material is one in which the melted form has low viscosity and the residue is solvent-extractable. It may also be advantageous for the melting temperature of the mandrel material to be well removed from degradation or ignition temperature of the material forming the mandrel. Certain materials contemplated to form the mandrel include, but are not limited to, polystyrene, cellulose acetate, vinyl acetate, and polyvinyl chloride. In certain embodiments, the mandrel is made of high impact polystyrene, which is removed, after electroforming and cutting, by melting at temperatures above 230° F. (110° C.), and in some embodiments melting at higher temperatures such as 430° F. (222° C.), followed by applying pressure at the proximal end of the pressure tube to drive the melted plastic out of the nozzle assembly 21. Thereafter, the nozzle assembly 21 may be rinsed with a solvent to complete removal of the mandrel material. Any removable material used in electroplating is potentially suitable for making a mandrel for practicing the invention. It should be noted that various gates (not illustrated, but known in the formation of plastic molds) may be used in the formation of the molds for the first and second mandrel portions 36, 52.

FIGS. 6A-6C illustrate the first and second mandrel portions 36, 52 coupled to the pressure tube 12 and the evacuation tube 16, ready for application of a thin uniform conductive coating before electroplating. In one embodiment, the conductive coating covers the region from distal tip 41 to a proximal limit P lying between planes 71 and 73, i.e., proximally of the tip region 60 of the second mandrel portion 52, and distally of the distal end of the hollow section 54 of second mandrel portion 52. Once the conductive coating is applied, the nozzle assembly 21 may be electroplated between distal tip 41 and at least about plane 73. Electroplating is continued to obtain the desired thickness of electroplated material. In one embodiment, a coating may extend more proximately, for example up to a plane 75 intersecting second mandrel portion 52.

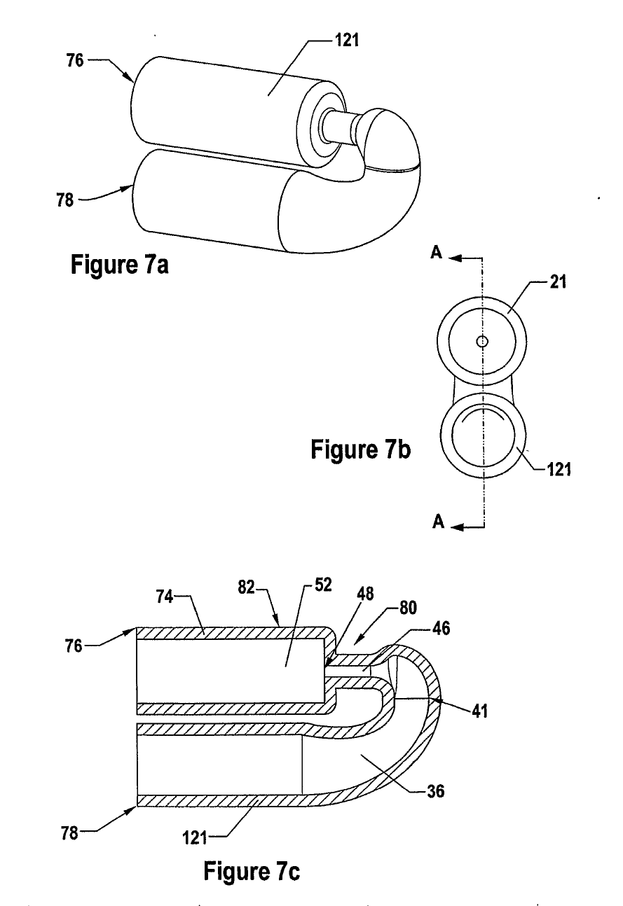

FIG. 7 illustrates the electroformed metal nozzle assembly 121 created by electroforming on a two portion mandrel system according to one embodiment of the present invention. The metal nozzle assembly 121 could, like nozzle assembly 21 of FIG. 4, be either formed in-situ (i.e with the mandrel portions attached to tubes 12 and 16) or formed separately. If formed separately, the ends 76 and 78 of the jet-forming assembly may be cut to produce metal-free ends, as shown. The metal layer may then be cut at a point 80 selected to lie in the right-circular collimating nozzle section 46 of the nozzle region, exposing tip region 48 of first mandrel portion 36. The region of second mandrel portion 52 between end 76 and cut 80 may be discarded, and the region from cut 80 to end 78 may become the nozzle assembly 121. In an in-place assembly with two mandrel portions, the cuts at 76 and 78 may be eliminated, with a second cut 82 made in addition to a cut at 80, to allow the second mandrel portion 52 and the overlying electroformed nozzle assembly 121 to be removed without distorting the nozzle end region at 80.

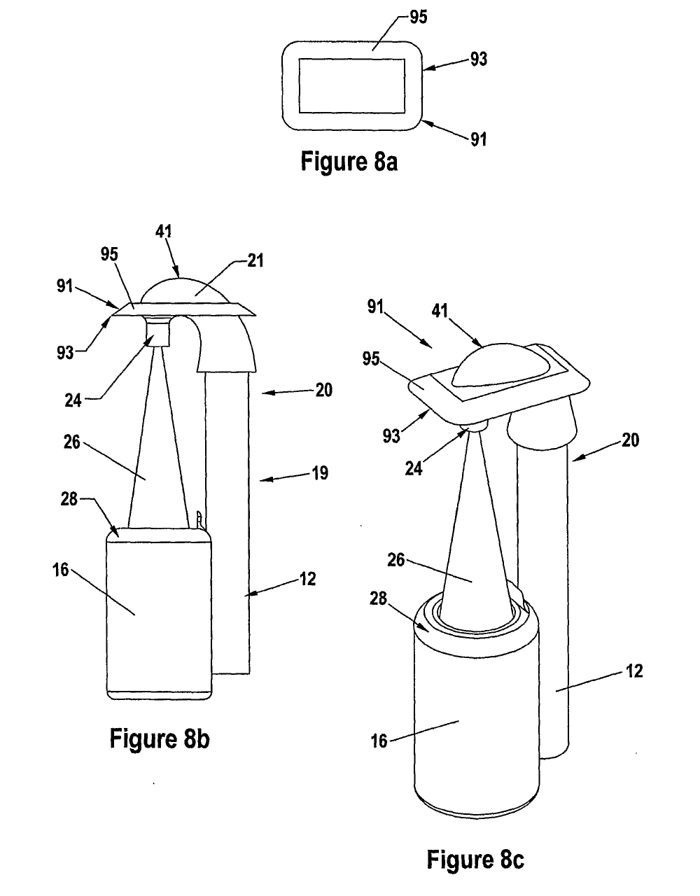

FIG. 8 is a schematic illustration of an electroformed nozzle assembly 21 of yet another embodiment of the present invention, where the nozzle assembly is shaped to include a tissue cutting surface 91. In this particular embodiment, the tip region 20 of the nozzle assembly 21 includes a scraping device 91. Other than the scraping device 91, the nozzle assembly may be configured to be similar to some of the above-described assemblies, having a distal end 41, a collimated nozzle region 24 adjacent a jet-opening 25. A liquid jet 26 may be emitted from the jet-opening 25 towards the jet receiving opening 28 of an evacuation tube 16. In this illustrative embodiment, the scraper 91 has an edge 93 and a sloping surface 95. As illustrated, the scraper 91 is coupled to the tip region 20 of the nozzle assembly 21, and may be retained there by conventional means, for example welding or brazing. In other embodiments, a tissue cutting surface may be formed integrally with the electroplated nozzle assembly. Tissue scraped away from the edge 93 may be drawn into jet beam 26 for maceration and removal.

A tissue cutting surface, such as the scraper 91, may also be formed integrally by placing a thin metallic foil at an appropriate location, and using it as a surface for formation of an electrodeposited layer. Post-electrodeposition machining may be used to refine the edge.

Besides a scraper 91, any of a variety of tissue manipulators can be affixed to the instrument of the invention and carried via the instrument to an operative site. These can include not only fixed devices with tissue cutting surfaces, such as scrapers 91, but more active devices which may include tissue cutting surfaces, such as forceps, scissor-type and other moveable cutters, distractors, and other elements of surgical or diagnostic devices, as described further below.

Yet another embodiment of the invention is shown in FIG. 9. In this embodiment, a mandrel 90 having a tip 92 is inserted in an outlet of a pressure tube 94 which has a U-shaped bend formed in it. The assembly has been coated with a thin conductive layer and then electroplated, forming a layer which forms the nozzle assembly 96. The nozzle assembly 96 and the mandrel tip 92 may be cut at C to form a jet-opening in the nozzle assembly. The residual material of mandrel 90 may then be removed, as described above. In some embodiments, an evacuation tube (not shown) may also be provided. A two-portion mandrel system may also be utilized, as the invention is not limited in this respect. In some embodiments, this configuration is less desirable because it may have a larger profile, for a given tube size. However, with some types of liquid jet surgical instruments, such as those described in US 2003/0125660, this may be a simple way to form a pre-aligned nozzle assembly attached to a pressure tube.

In the embodiments of FIGS. 9 and 1, the liquid jet is directed proximally. Alternate directions of the jet beam (not illustrated) are contemplated, including distal tips in which the liquid jet is directed distally (for example, as in FIG. 9, but without a bend in the tube), or tips emitting jets laterally, i.e., at some angle other than distally (0 deg.) or proximally 180 deg. (FIG. 9, FIG. 1), such as approximately 45 deg., approximately 60 deg., approximately 75 deg., approximately 90 deg., approximately 120 deg., or other angles.

FIGS. 10A and 10B illustrate first and second mandrel portions used to electroform a nozzle assembly according to yet another embodiment. In this embodiment, the first mandrel portion 36 is similar to the above described first mandrel portions 36 (e.g., see FIGS. 6A-6C). The second mandrel portion 152 is formed to have some similar design features as first mandrel portion 36, in that it has a section 138 which may fit securely inside evacuation tube 16, and a section 140 extending distally of tube 16 and positioned by step flange 139. Section 140 is analogous to section 60 of mandrel 52 (see, e.g., FIG. 5A), and has a central bore 166 into which tip 48 of nozzle region 46 of first mandrel 36 may fit. As with mandrel 52, mandrel 152 may have a second hole 168 for clearing debris.

The embodiment shown in FIGS. 10A-10B may be rendered conductive distally of a selected point H1, which is selected to prevent electroforming of the proximal region of section 140 of mandrel 152, while allowing electroforming of the rest of the exposed portions of the mandrels 36 and 152, and the distal end of tube 12. After electroforming the nozzle assembly (not illustrated), cuts may be made at points C1 and C2. The cut at C1 may remove the proximal end of tip 48 which may form a jet-opening in the nozzle assembly, and the cut at C2 may be made slightly distally of the end of tube 16. The material or materials of the mandrels is or are removed, and the finished device may be ready for use.

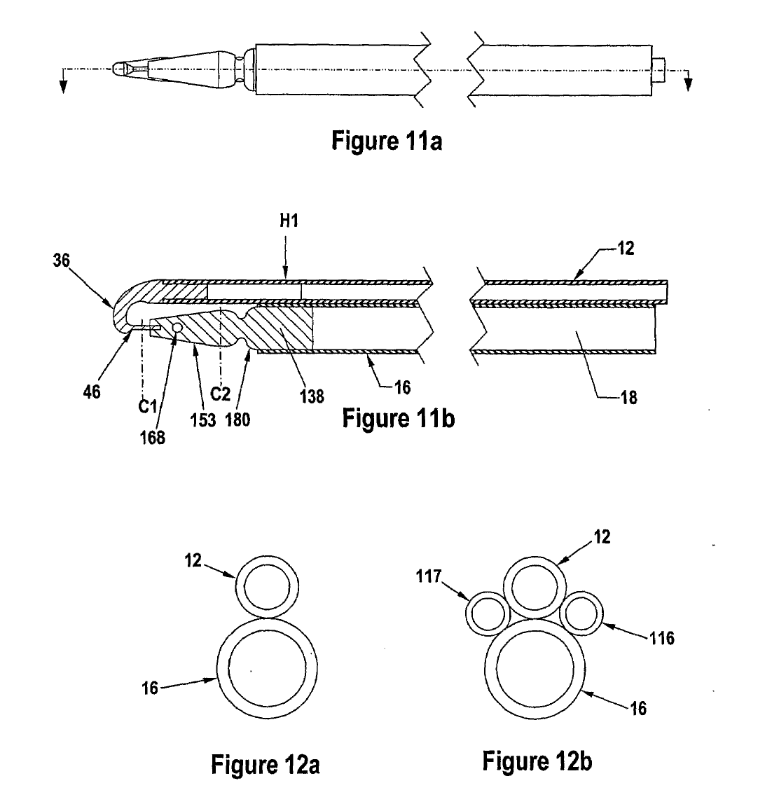

FIG. 11 illustrates another embodiment in which the second mandrel portion 153 is configured to provide a constriction in the evacuation path via evacuation tube 16. The components may be rendered conductive distally of point H1, which may permit electroforming of part of evacuation tube 16. Second mandrel portion 153 may be tapered distally, and has an indentation, such as a groove 180. The tapering may reduce affixation of the proximal regions of the second mandrel portion 153 to the pressure tube 12 during electroforming. The groove 180 provides a size-limiting aperture in the flow path of evacuation tube 16 after electroforming.

After electroforming, the assembly may be cut at points C1 and C2. The remaining materials of the mandrels are removed. The cuts result in the functional extension of tube 16 but with a constriction, at the location of groove 180, limiting the diameter of the lumen 18 of tube 16. This may help create a significant stagnation pressure when the jet beam enters tube 16, which assists in evacuation of liquids and maceration of solids present at the site of use of the device. It should be recognized that in addition to a groove 180, other types of indentations, such as, but not limited to one or more dimples or recesses may also be formed into the second mandrel portion 153 to create a constriction in the evacuation tube 16.

In yet another aspect of the invention, access to the operative site may be provided by the creation of passages or holes in the electroformed nozzle assembly of the device. Any of a variety of conventional devices can be placed in such passages. In certain embodiments, these passages are open to the environment at the distal tip region of the instrument when in operation in the body.

FIG. 12B illustrates one embodiment of this feature of the invention. A cross section of the pressure tube 12 coupled to the evacuation tube 16 is shown in FIG. 12A. In FIG. 12B, two additional tubes, first tube 116 and optional second tube 117, are added to the assembly to form two passages which extend along the pressure and/or evacuation tubes. These auxiliary tubes may be electroformed about a mandrel to provide passages adjacent the pressure tube. The tubes 116 and 117 may extend proximally of the operative electroformed nozzle assembly, and may extend either directly, or via junctions with other tubes, to the proximal end of the instrument. In some embodiments, the tubes 116 and/or 117 may be formed during the electroforming of the nozzle assembly. For example, in one embodiment, the tubes 116, 117 may be formed by solid extrusions of materials used as mandrels, which are removable after electroforming. In one embodiment, when the tubes 116 and/or 117 are formed with pre-made tubes, for example of stainless steel, the tubes may be plugged at their distal ends with pins of mandrel material (not illustrated) during the electroforming process. These pins may extend distally of the tubes and may be cut after electroforming of the tip to allow holes to be present in the electroformed tip communicating with the tubes 116 and 117.

In another embodiment, the tubes 116 and/or 117 may be formed with cylinders or other elongated shapes of extractable material. These may be attached to the evacuation tube 16 and/or the pressure tube 12, and then after electroforming, exposed sufficiently at the distal end to be removed in the same manner as the mandrels, or by a different procedure appropriate for the material of the tubes 116, 117.

A variety of devices are contemplated for use with these passages created by tubes 116, 177. Such devices include, but are not limited to, fiber optics, for emitting light and/or collecting images; cables, for example driving attached devices such as forceps; probes for diagnostics (pH, pO2, electrodes, etc.); and sources of electric current or voltage, such as electrocautery probes, or electricity supply for other devices. One or more of the passage in the tubes 116, 117 may supply air, vacuum, water, saline, contrast fluid, or pharmaceutically effective agents, to the site of the operation. Any of these functions can be supplied either through an embedded tube 116, 117, or by a separate supply feeding one or more holes in the electroformed nozzle assembly, formed as described above.

It should be recognized that although an embodiment having one or two passages in tubes 116, 117 has been described, more apertures or tubes may be provided as the invention is not limited in this respect. It should be recognized that in some embodiments, the more passages may increase the profile of the surgical instrument (e.g., cross sectional area) and decrease flexibility, while tubes including passages can weaken the tip. Thus, in certain embodiments, the number of passages is limited to the number of passages needed for a particular surgical procedure.

Another aspect of the inventions involves the performance of surgical or medical procedures on patients using the inventive surgical instruments fabricated as described above. In one embodiment, the invention provides a method of performing a medical or surgical procedure on a patient that involves supplying a liquid at a pressure of at least 1000 psig, in certain cases at least 2000 psig, at least 5000 psig, at least 10000 psig, at least 15000 psig, at least 30000 psig, or at least 50000 psig to the pressure tube of a liquid jet surgical instrument manufactured by an inventive electroforming method as described above, creating a liquid jet with the instrument, and directing the liquid jet at a tissue of the patient to cut, ablate, pulverize and/or debride the tissue. In certain such embodiments, the method further comprising removing liquid comprising the liquid jet and tissue removed from the patient by the jet from a surgical site to a proximal end of the evacuation tube using only the stagnation pressure generated by the liquid jet and without the need for an external source of suction applied to the evacuation tube.

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention. All definitions, as defined and used herein, should be understood to control over dictionary definitions, definitions or usage in documents incorporated by reference, and/or ordinary meanings of the defined terms.

It should also be understood that, unless clearly indicated to the contrary, in any methods claimed herein that include more than one step or act, the order of the steps or acts of the method is not necessarily limited to the order in which the steps or acts of the method are recited.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e., to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

What is claimed is:

1. A method of manufacturing a liquid jet-forming surgical instrument comprising a pressure tube, an evacuation tube and a nozzle, the method comprising:
electroforming a nozzle assembly of the surgical instrument on a mandrel, wherein the nozzle assembly includes at least one nozzle providing a jet-opening, wherein the nozzle is shaped to form a liquid jet as a liquid at high pressure flows therethrough; removing the mandrel from the nozzle assembly;
coupling an outlet of the pressure tube of the surgical instrument to the nozzle assembly; and positioning an inlet of the evacuation tube of the surgical instrument such that a jet-receiving opening of the evacuation tube is located opposite the jet-opening of the nozzle to enable the evacuation opening to receive the liquid jet, when the instrument is in operation.

2. A method as in claim 1, further comprising:
coupling the outlet of the pressure tube of the surgical instrument to the mandrel; and
electroforming the nozzle assembly of the surgical instrument on the mandrel so that the nozzle assembly is integrally connected to the outlet of the pressure tube.

3. A method as in claim 1, further comprising: inserting the mandrel into the outlet of the pressure tube before the electroforming act.

4. The method of claim 1, wherein the mandrel includes at least a first mandrel portion and a second mandrel portion.

5. The method of claim 4, further comprising:
coupling the first mandrel portion to the outlet of the pressure tube; and coupling the second mandrel portion to the inlet of the evacuation tube.

6. The method of claims 1, further comprising:
cutting the nozzle assembly to form the jet-opening.

7. The method of claim, further comprising:
coating at least a portion of the mandrel and at least a portion of the pressure tube with an electroconductive material before the electroforming act.

8. A method as in claims 1, wherein the evacuation tube is immobilized with respect to the pressure tube.

9. A method as in claim 8, wherein the evacuation tube is coupled to the pressure tube.

10. A method as in claim 9, wherein the evacuation tube is coupled to the pressure tube before the electroforming act.

11. A method as in claims 1, wherein the electroforming act continues until the thickness of the wall of the nozzle assembly is at least 0.125 millimeters.

12. A method as in claims 1, wherein the nozzle assembly is configured to include a tissue cutting surface.

13. A method as in claims 1, wherein the nozzle assembly is formed to include a collimated nozzle region adjacent the jet-opening.

14. A method as in claim 1, wherein during the electroforming act, at least one auxiliary tube is electroformed to provide a passage adjacent the pressure tube.

15. (canceled)

16. (canceled)

17. A method of manufacturing a liquid jet-forming surgical instrument comprising a pressure tube, an evacuation tube and a nozzle, the method comprising:
coupling a first mandrel portion to an outlet of the pressure tube of the surgical instrument;
coupling a second mandrel portion to an inlet of the evacuation tube of the surgical instrument, wherein the second mandrel portion is constructed and arranged to be coupled to the first mandrel portion;
electroforming a nozzle assembly of the surgical instrument on the first and second mandrel portions;
cutting the nozzle assembly to create at least one nozzle providing a jet-opening, wherein the nozzle is shaped to form a liquid jet as a liquid at high pressure flows therethrough, wherein a jet-receiving opening of the inlet of the evacuation tube is located opposite the jet-opening of the nozzle to enable the evacuation opening to receive the liquid jet, when the instrument is in operation; and
removing the first and second mandrel portions from the nozzle assembly.

18. A method as in claim 17, further comprising:
coating at least a portion of the first and second mandrel portions with an electroconductive material before the electroforming act.

19-21. (canceled)

22. A method as in claim 17, wherein the first mandrel portion is substantially U-shaped.

23. A method as in claim 17, wherein the first mandrel portion includes a first end and a second end, where the first end is inserted into the pressure tube and the second end tapers to a tip end.

24. A method as in claim 17, wherein the first mandrel portion is substantially U-shaped.

25. A method as in claim 17, wherein the a longitudinal axis of the pressure tube is substantially parallel to the a longitudinal axis of the evacuation tube.

26. (canceled)

27. (canceled)

28. A method of claim 17, further comprising removing a portion of the nozzle assembly surrounding the second mandrel portion.

29-36. (canceled)

37. A liquid jet-forming surgical instrument manufactured according to the method of claim 1.

38. A liquid jet-forming surgical instrument manufactured according to the method of claim 17.

39. A use of the jet-forming surgical instrument manufactured according to the method of claim 1 for excision of tissue comprising:
contacting a tissue with a liquid jet of the jet-forming surgical instrument, and excision the tissue with the liquid jet of the jet-forming surgical instrument.
